(19) United States
(12) Patent Application Publication
(10) Pub. No.: US 2010/0129784 A1
(43) Pub. Date: May 27, 2010

(54) PERFUSION DEVICE FOR HOLLOW ORGANS, AND THE USE THEREOF FOR THE PERFUSION OF AN EXPLANTED HOLLOW ORGAN

(75) Inventors: Bertram Eichentopf, Bad Soden (DE); Walter Klatte, Bad Homburg (DE); Ulrike Wippermann, Obertshausen (DE); Uwe Bauer, Ebersberg (DE); Stephan Nees, Muenchen (DE)

Correspondence Address: COLLARD & ROE, P.C., 1077 NORTHERN BOULEVARD, ROSLYN, NY, 11576, US

(73) Assignee: BIOTEST AG, Dreieich (DE)

(21) Appl. No.: 12/452,298

(22) PCT Filed: Jun. 23, 2008
(86) PCT No.: PCT/EP2008/057955

§ 371 Date: Dec. 23, 2009

(30) Foreign Application Priority Data
Jun. 25, 2007 (DE) 10 2007 029 150.9

(51) Int. Cl. A01N 1/02; A61M 39/24

(52) U.S. Cl. 435/1.2; 604/247

(57) ABSTRACT

A perfusion liquid is suctioned from a perfusion liquid reservoir (16) through the automatic two-way valve (4) by drawing a syringe (5) serving as the perfusion liquid displacement device. For this purpose, the connection from the syringe (5) to the cannula (14) is closed by the two-way valve (4). The two-way valve (4) closes the connection to the perfusion liquid reservoir due to the pressure increase during the insertion of the syringe plunger and opens the connection from the syringe (5) to the cannula (14). A pressure relief valve (7) is attached at the T piece (6), exposing a discharge line (8) in case a threshold pressure is exceeded. A check valve (11) is attached at the extension of the cannula (14), preventing the backflow from the cannula (14) into the remaining system.

The present invention relates to a perfusion device for hollow structures, particularly organic ones, particularly to a perfusion device in accordance with the preamble of claim 1, as well as its use, particularly for perfusion of an explanted hollow organ.

Such perfusion devices can be used, for example, in connection with heart surgery operations. In the case of heart bypass operations, explanted vein or artery segments are rinsed before reimplantation in the heart, and are exposed to a certain pressure after ligature of the branchings, in order to check them for leaks. In order to prevent damage to the endothelium of the segments, it is necessary to carry out the rinsing with a limited pressure, and to use a suitable rinsing solution for rinsing. Both excess pressure stress and a non-physiological medium can permanently damage the endothelium and prejudice thrombogenic processes.

The use of endothelium-protective perfusion solutions is described in the German Offenlegungsschrift [unexamined patent published for public scrutiny] DE 103 26 764 A1. The chamber-like perfusion device furthermore disclosed in this reference is very specifically designed for endothelium preservation, and cannot be used for many other perfusion processes in hollow structures.

A perfusion device of the type mentioned initially, which is used for rinsing explanted vein sections, is known from the reference EP 0 032 826 A2. The known perfusion device has a perfusion fluid syringe for supplying the perfusion fluid, and a vein cannula as well as a balloon-like element interposed between them, which serves as a pressure limiter. In this connection, restriction of the pressure is predetermined by the elastic properties of the balloon sheath, which avoids pressure peaks even in the event of rapid and forceful activation of the perfusion fluid syringe, by expanding. A shut-off cock is disposed between balloon element and perfusion fluid syringe.

The previously known perfusion device has the disadvantage that the amount of fluid that a syringe can hold is, of course, limited, and this leads to the result, in the event of a longer rinsing period, i.e. a larger amount of rinsing fluid that is required, that the perfusion fluid syringe has to be replaced during use, if necessary even multiple times. This procedure is complicated, particularly since not only does the syringe have to be removed and set on again, but also, in this connection, the shut-off cock first has to be closed each time and then opened again. Furthermore, there is the risk of pinching the balloon element or otherwise exerting pressure on it from the outside. This can have the result that the balloon element can no longer exert its pressure-limiting effect correctly, or that pressure peaks are actually produced by means of the effect of external pressure on the balloon sheath, which can lead to bursting or damage of the rinsed vein.

In view of the requirements that must fundamentally be established for a perfusion device, and the disadvantages of the known state of the art, the present invention is based on the task of allowing convenient and reliable handling of a perfusion device of the type stated initially, even in the case of long perfusion times or large amounts of perfusion fluid that are required, and of guaranteeing a reliable pressure restriction. Furthermore, it is supposed to be possible to use the invention in many different ways.

According to one aspect of the present invention, this task is accomplished by means of a perfusion device for hollow structures, particularly organic ones, which has a perfusion fluid displacement device (which can be implemented as a syringe or also in some other way), a cannula, a multi-way valve device disposed between perfusion fluid displacement device and cannula, and a perfusion fluid feed line connector, whereby a hydraulic connection between the perfusion fluid displacement device and the perfusion fluid feed line connector as well as between the perfusion fluid displacement device and the cannula can be produced.

According to a particularly preferred further development of the invention, the perfusion device has at least one excess pressure limiter between the perfusion fluid displacement device and the cannula, whereby at least one pressure relief valve is disposed in the excess pressure limiter. If a threshold pressure is exceeded, the pressure relief valve automatically opens a drain opening in order to discharge the excess pressure toward the outside.

According to another particularly preferred further development of the invention, the valve device is disposed between the perfusion fluid displacement device and the excess pressure limiter, so that a hydraulic connection between the perfusion fluid displacement device and the excess pressure limiter can be produced by way of the valve device.

In this connection, a continuous fluid connection can be understood to be a hydraulic connection. Organic hollow structures are understood to be, in particular, also hollow organs, blood vessels, and parts of them.

Such a perfusion device can not only be advantageously used for rinsing, i.e. irrigation of explanted blood vessels, but can also be used, in practical manner, for many other applications in which a fluid is supposed to be filled into hollow organic structures or conveyed by these, for example in the treatment of vascular diseases, in transfusion medicine, in intensive-care medicine, in (plastic, cardiac, vascular) surgery (for example in bypass operations), particularly within the scope of implantations, reimplantations, amputations, restorative operations, e.g. after amputations, when suturing on body parts that have been cut off, and more of the like.

Perfusion devices according to the invention can advantageously be easily implemented in that they are transportable, as a whole or broken down into their essential parts, and can be kept on hand in mobile emergency kits and the like, for example.

As compared with the previously known perfusion device described above, the present invention has the significant advantage that even large amounts of perfusion fluid can be passed through, without the perfusion fluid syringe (or other perfusion fluid displacement device) having to be pulled off and reconnected, since the perfusion fluid feed line connector permits (fundamentally almost unlimited) feed of perfusion fluid.

Furthermore, a pressure relief valve, as compared with a balloon element, offers the advantage of greater functional safety in connection with the risk of pinching. In contrast to a balloon element, in which the internal pressure progressively increases with increasing expansion, a clearly defined limit pressure can furthermore be set with a pressure relief valve, which triggers draining of fluid, as a threshold pressure.

Alternatively, it is also possible to do without the excess pressure limiter in the pressure system of the perfusion device, if a manometer device having a display is used. The personnel operating the device can then control the pressure during perfusion themselves.

As has already been indicated, the perfusion fluid displacement device is preferably configured in the form of a syringe. Depending on the requirements of a concrete application, this can be configured in different ways, for example with scaling, as a disposable syringe, glass syringe, plastic syringe, syringe with integrated filter, etc.

According to a particularly preferred further development of the invention, a multi-way valve device is provided, which automatically releases the hydraulic connection between perfusion fluid syringe and perfusion fluid feed line connector when the perfusion fluid syringe is filled, and automatically closes it when the perfusion fluid syringe is pressed and releases the path in the direction of the hollow structure to be perfused. The need for manual valve activation is then eliminated, thereby further simplifying use of the device and making it easier for the user.

If the perfusion fluid displacement device is structured not as a syringe, but in some other way, for example as a balloon pump, it is also advantageous to provide a multi-way valve device that automatically opens and closes the perfusion fluid feed line connector.

However, a manually operated multi-way valve, for example a simple two-way cock, is also possible.

Particularly preferably, the perfusion device according to the invention has a perfusion fluid reservoir unit connected with the perfusion fluid feed line by way of the perfusion fluid feed line connector, in which unit perfusion fluid that is present can preferably be tempered. Here, depending on the application, both simple heating and/or cooling devices as well as more complicated thermostat devices can be advantageous; the latter allow tempering in a predetermined narrow temperature range.

Regulated or also non-regulated tempering opens up additional application fields, such as preservation (use of cooled perfusion fluid), thawing and warming of hollow structures, for example in connection with temporary, particularly also medium-term or term storage of blood vessels, organs, and body parts. After all, structures intended for reimplantation or also body parts intended to be sutured on again are sometimes frozen or stored on ice, and have to be warmed up before the corresponding operation takes place. In this connection, it can be noted that devices according to the invention are also suitable as equipment in ambulances, for example to perfuse separated body parts with a suitable perfusion fluid, directly on location (for the purpose of preservation/stabilization).

It is advantageous to structure the perfusion fluid feed line as a hose. Depending on the case of use, fundamentally different materials are possible here, as is also true for other components of devices according to the invention. (Depending on the function of the individual component, and whether or not it is to be structured as a disposable or multi-use part, biocompatibility, autoclavability, and pressure stability can serve as selection criteria for the individual materials to be used.)

Fundamentally, the perfusion fluid reservoir unit can be filled with the most varied perfusion fluids, adapted to the case of use, in each instance. Depending on the application in question, water, whole blood, blood plasma, blood plasma fractions, buffer solutions, saline solutions (e.g. sodium chloride solution, physiological electrolyte solutions), nutrient media, solutions of plasma proteins (e.g. compositions that contain human albumin and/or blood replacement substances, all the way to artificial blood) are possible. The use of endothelium protection solutions, which have been commercially available until now under the trade name BISEKO, are particularly preferred for use with explanted blood vessels.

It is particularly advantageous to equip the perfusion fluid reservoir unit with a container holder into which a perfusion fluid container can be set. In this manner, in accordance with the cartridge principle, rapid container replacement and thus quasi-continuous feed of almost any desired amounts, however large, of perfusion fluid, also fluid that changes in terms of its composition, can be implemented. Container holders into which multiple perfusion fluid containers can be inserted at the same time can also be advantageous. Thus, “switching” from one to another container can be implemented by means of suitable change-over valve devices and/or multi-way valve devices.

It is advantageous if the perfusion fluid reservoir unit has a hollow perforation mandrel, preferably configured with two channels, by way of which a hydraulic connection between the perfusion fluid container and the perfusion fluid feed line can be produced by means of inserting it into a perfusion fluid container equipped with a suitable septum or one that can be perforated, as a whole.

According to a preferred embodiment, the container holder has a locking mechanism with which a perfusion fluid container that has been inserted can be locked in place in the container holder. The locking mechanism can advantageously have a thread, particularly a multi-channel thread, with an engagement mechanism, for example a saw-tooth thread.

For simple set-up or fastening at the location of use, the perfusion fluid reservoir unit can be provided with a stand or a hanging device.

Depending on the perfusion fluid used and the conditions of use provided, it is advantageous if the perfusion fluid reservoir unit has a filter device.

According to another preferred embodiment, the perfusion device has a collecting container that stands in a hydraulic connection with the drain opening, for example by way of a suitable hose.

It is advantageous if barrier means for shutting off the discharge through the drain opening are also provided; these can comprise a hose piece with a hose clamp or a cock, for example.

According to an advantageous further development of the invention, the excess pressure limiter can be designed for opening a drain opening at at least two different threshold pressures, in each instance. This can be advantageously implemented, for example, in that the excess pressure limiter is equipped with two pressure relief valves, each having a threshold pressure that is different from the other pressure relief valve.

If one switches the pressure relief valves in parallel and ensures that at least one of the pressure relief valves can be shut off, an advantageous embodiment is obtained, in which the threshold pressure of the respective pressure relief valve that is not shut off is the pressure that causes perfusion fluid to be drained if it is exceeded. In this connection, shut-off can be implemented, as mentioned above, by means of a hose clamp or a cock. However, pressure relief valves having an integrated shut-off function or a serial circuit of a pressure relief valve and stopcock are also possible.

Using such an embodiment, it is possible to manage complex applications that require different pressures. For example, an explanted blood vessel set onto the cannula can thus be irrigated at a lower maximal pressure, and after the end of the blood vessel that has been removed from the cannula has been sutured on at the intended placement location, a test for leaks can be carried out at a higher pressure.

Preferably, the lower of the two threshold pressures lies in a range of 20 to 200, particularly preferably 50 to 150 Torr, and the higher of the two threshold pressures lies in a range of 100 to 700, particularly preferably 250 to 350 Torr. 100 Torr for irrigation and 300 Torr for the leak test can serve as a guideline value for explanted veins.

According to a particularly preferred embodiment of the invention, a manometer device can be provided in the device for checking the current pressure, as has already been mentioned above.

It is advantageous if the manometer device is disposed between the cannula and the perfusion fluid displacement device, to measure the pressure in the pressure system. In this way, it is possible for the personnel operating the perfusion device to obtain values of the current pressure or pressure increase in the pressure system. At the same time, pressure can be relieved by way of a drain opening, by means of a pressure relief valve, if a threshold pressure is exceeded.

According to another preferred further development, the manometer device of the perfusion device is disposed between the cannula and the valve device to measure the pressure in the pressure system.

Furthermore, the manometer device can have a piezo pressure sensor, whereby other embodiments are also possible. Preferably, the manometer device can have a display for indicating the pressure applied at the manometer device, whereby the display of the manometer device is preferably a digital display.

Furthermore, the manometer device (20) can have an alarm device that triggers a signal if an early warning pressure is exceeded. In this connection, the signal can be issued acoustically, for example in the form of a tone, or also optically/visually, for example by means of a light-emitting diode.

According to a particularly preferred embodiment, the device can have a modular construction and can be broken down into parts, preferably without tools. For one thing, the transportability already addressed above can be improved in this way; for another, in this way it is easy to combine disposable components and multi-use components with one another.

Particularly preferably, the following components are replaceable: the perfusion fluid syringe or other perfusion fluid displacement device, the cannula, the excess pressure limiter or parts of it, for example to change a threshold pressure, the perfusion fluid feed line, and the multi-way valve, for example in order to tune its response behavior, if this is an automatic multi-way valve.

In many cases, it is particularly important to easily remove and reconnect the cannula. In this manner, for example, a hollow structure can be stored together with the cannula, and can easily be connected to the perfusion device again after the end of the storage period. Furthermore, different hollow structures can require the use of very different cannulae, particularly with regard to their diameter, but also their configuration.

In order to make rapid and easy, destruction-free and/or tool-free replacement of individual components, different connection means, which are known per se, can be provided, such as Luer lock connections, bayonet closures, snap closures, different quick-connection threads, clamp connections, plug-in connections, and more of the like.

According to another preferred embodiment, the perfusion device has a check valve to prevent back-flow through the cannula into the perfusion device. This can also be configured to be replaceable.

As indicated above, the cannula can have the most varied configurations, depending on the purpose of use, for example also as in the conventional device described above. It is advantageous if the cannula has a fastening device for fixing the hollow organ in place on the cannula and/or can be configured conically at least in certain sections. In this connection, expanding and narrowing outside diameters as well as combinations of them are possible.

Unless something different is explained, in an individual case; commercially available standard components or embodiments based on them can be used for almost all the components, particularly valves, hoses, T-connections, connectors, cannulae, syringes, and clamps.

Fundamentally, any variant of the invention described and/or indicated within the scope of the present application can be particularly advantageous, depending on the economic and technical conditions in an individual case. Unless something to the contrary is stated, and to the extent that this is fundamentally technically feasible, individual characteristics of the embodiments described can also be interchanged or combined with one another.

In the following, examples of preferred embodiments of the present invention will be explained in greater detail, using the related drawings.

In this connection, the drawings are schematic and, for reasons of clarity, are not precisely representations to scale. In particular, ratios of the dimensions relative to one another can deviate, in some cases greatly, from actual embodiments.

Elements that correspond to one another have been provided with the same reference symbols in the individual figures, to the extent that this makes sense.

The figures show:

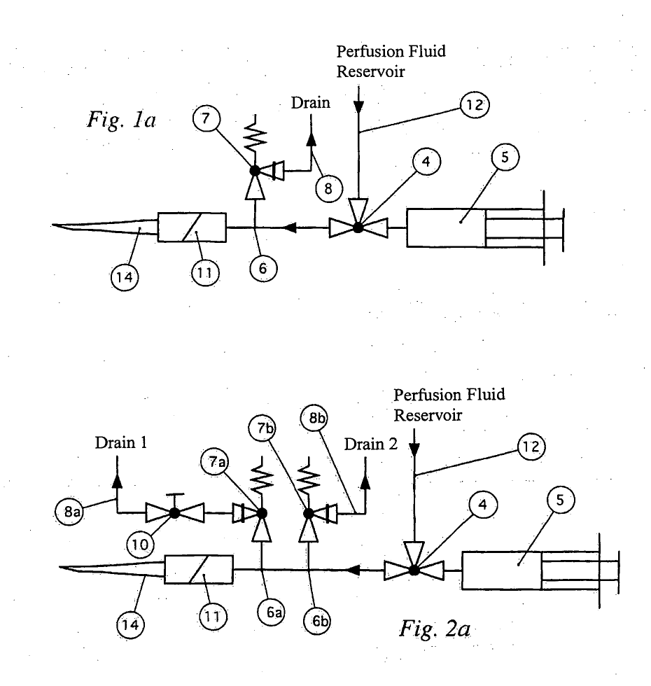

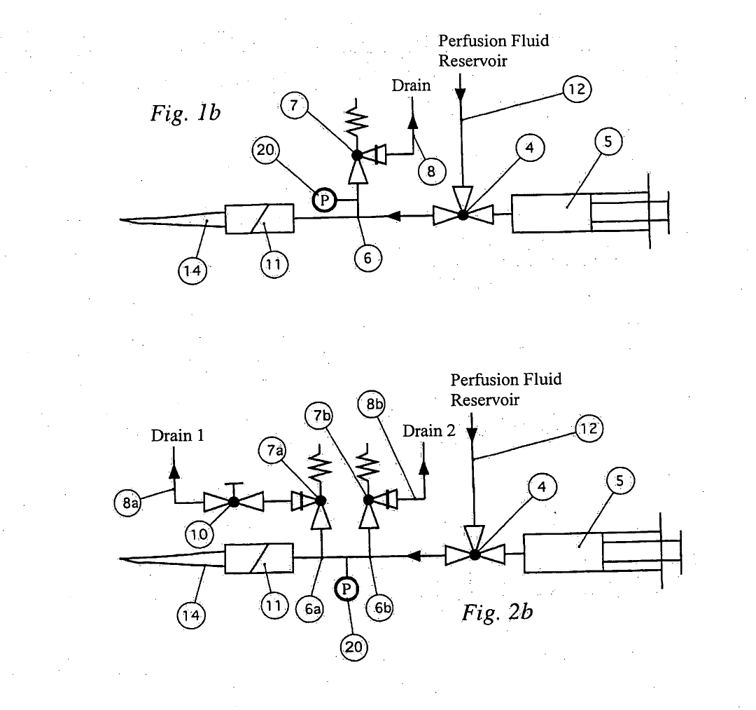

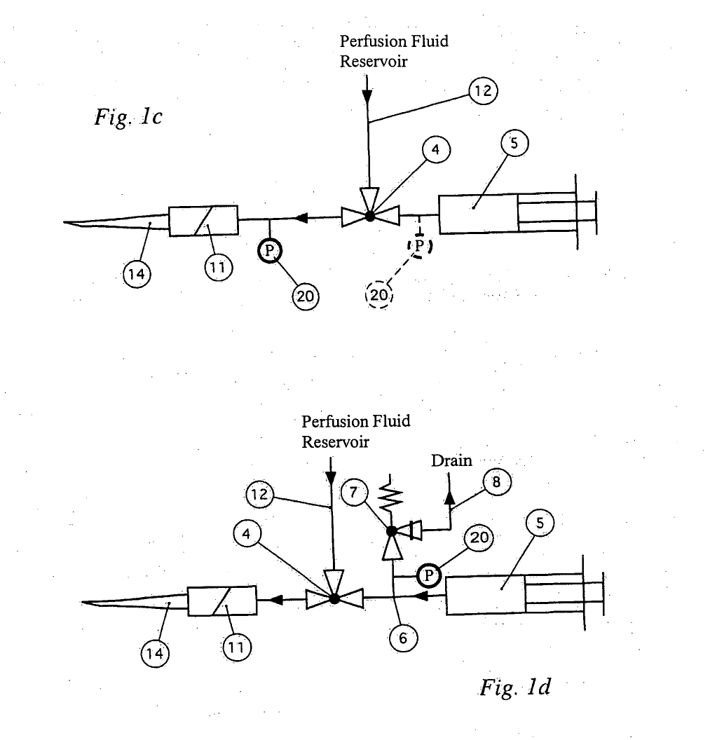

FIG. 1a a purely schematic flow chart of the basic structure of an embodiment of the perfusion device according to the invention, with a pressure relief valve,

FIG. 1b a purely schematic flow chart of another embodiment of the perfusion device according to the invention, with a pressure relief valve and a manometer device,

FIG. 1c a purely schematic flow chart of another embodiment of the perfusion device according to the invention, with a manometer device without a pressure relief valve,

FIG. 1d a purely schematic flow chart of another embodiment of the perfusion device according to the invention, with a pressure relief valve and a manometer device,

FIG. 2a a purely schematic flow chart of the basic structure of an embodiment of the perfusion device according to the invention, with two pressure relief valves,

FIG. 2b a purely schematic flow chart of another embodiment of the perfusion device according to the invention, with two pressure relief valves and a manometer device,

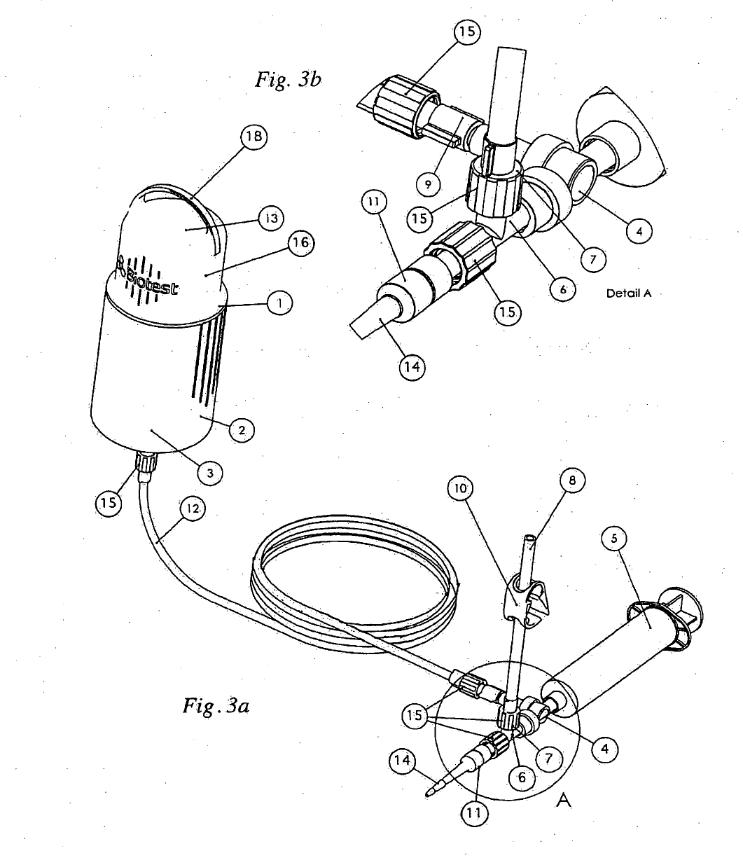

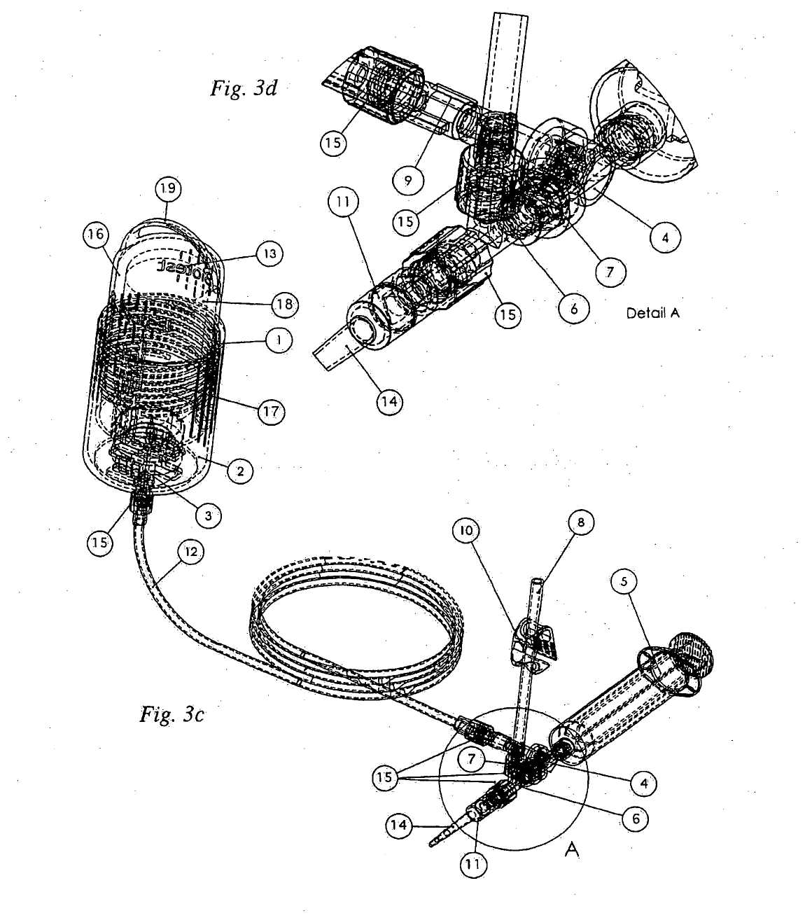

FIG. 3a a perspective overall view of an embodiment of the perfusion device according to the invention, having a structure similar to that in FIG. 1,

FIG. 3b a perspective partial view A of the embodiment from

FIG. 3a, enlarged,.

FIG. 3c the view from FIG. 3a, but with a visible representation of hidden edges as broken lines, and

FIG. 3d the view from FIG. 3b, but with a visible representation of hidden edges as broken lines.

FIG. 1a, as an exemplary embodiment, shows the flow chart of a perfusion device according to the invention, with a pressure relief valve 7. By means of filling the syringe 5 that serves as the perfusion fluid displacement device, perfusion fluid is drawn in from a perfusion fluid reservoir (not shown) by way of the perfusion fluid feed line 12, by means of the automatic two-way valve 4. In this connection, the connection from the syringe 5 to the cannula 14 is closed by means of the two-way valve 4. By means of the pressure increase when the syringe plunger is pushed in, the two-way valve 4 closes the connection to the perfusion fluid reservoir and opens the connection from the syringe 5 to the cannula 14. A pressure relief valve 7 is set onto the T-piece 6, which valve releases a drain line 8 if a limit pressure is exceeded, for example 100 Torr for irrigation of a vein segment connected with the cannula 14. A kick-back valve 11 is affixed at the back end of the cannula 14, which prevents back-flow from the cannula 14 into the rest of the system.

The flow chart shown in FIG. 1b shows another exemplary embodiment of a perfusion device according to the invention, with a pressure relief valve 7 and an additionally provided manometer device 20. The structure of the perfusion device essentially corresponds to the structure of the arrangement from FIG. 1a, whereby the manometer device 20 is disposed between the T-piece 6 and the pressure relief valve 7.

FIG. 1c, as another exemplary embodiment, shows the flow chart of a perfusion device according to the invention, with a manometer device 20. The exemplary embodiment does without an excess pressure limiter. The manometer device 20 measures the current pressure in the system of the perfusion device, between the valve device 4 and the cannula 14. Furthermore, it is possible to dispose the measurement location of the manometer device in the hydraulic connection between the perfusion fluid displacement device 5 and the valve device 4 (broken line in FIG. 1c).

The flow chart shown in FIG. 1d shows another exemplary embodiment of a perfusion device according to the invention, with a pressure relief valve 7 and an additionally provided manometer device 20. In this connection, the pressure relief valve 7 is disposed between the perfusion fluid displacement device 5 and the valve arrangement 4. The manometer device 20 is switched ahead of the pressure relief valve 7, to measure the pressure.

The flow chart shown in FIG. 2a shows an exemplary embodiment with two pressure relief valves 7a and 7b, which are set on by way of corresponding T-pieces 6a and 6b. The first threshold pressure, as of which the first pressure relief valve 7a releases the first drain line 8a, is lower than the second threshold pressure, as of which the second pressure relief valve 7b releases the second drain line 8b. If the first drain line 8a is shut off by way of the shut-off device 10 that can be configured as a shut-off cock, blind stopper, or hose clamp, for example, then the second threshold pressure is the decisive limit pressure that can be reached, with the perfusion fluid being discharged by way of the second drain line 8b if this pressure is exceeded. On the other hand, if the shut-off device 10 remains open, then the first threshold pressure is the decisive limit pressure that can be reached, with the perfusion fluid being discharged by way of the first drain line 8a if this pressure is exceeded. Otherwise, the device functions as described using FIG. 1. By means of filling the syringe 5 that functions as the perfusion fluid displacement device, perfusion fluid is drawn in from a perfusion fluid reservoir (not shown), by way of the perfusion fluid feed line 12, by means of the automatic two-way valve 4. The connection from the syringe 5 to the cannula 14 is closed by means of the two-way valve 4. By means of the pressure increase when the syringe plunger is pushed in, the two-way valve 4 closes the connection to the perfusion fluid reservoir and opens the connection from the syringe 5 to the cannula 14.

Using the device shown in FIG. 2a, two different limit pressures can therefore be implemented by means of simply opening and closing the shut-off device 10, and this allows use for irrigation at low pressures and a subsequent leak test at higher pressures, for example.

FIG. 2b shows, as an exemplary embodiment, a flow chart with two pressure relief valves 7a and 7b, which are set on by way of corresponding T-pieces 6a and 6b. Furthermore, the exemplary embodiment additionally has a manometer device 20. The structure of the perfusion device essentially corresponds to the structure of the arrangement from FIG. 2a, whereby the manometer device 20 is between the T-pieces 6a and 6b, in order to indicate the current pressure in the pressure system of the perfusion device.

In this connection, the perspective views in FIG. 3a-d show a perfusion device structured in accordance with the flow chart shown in FIG. 1, which can be used, for example for irrigating an explanted vein segment within the scope of a heart bypass operation. In this connection, FIGS. 3b and 3d show the detail marked as A in FIGS. 3a and 3c as a detail view, in slightly enlarged form. In FIGS. 3c and 3d, hidden edges are shown as broken lines.

The perfusion device is implemented as a modular system, whereby individual elements are connected with one another by way of a Luer lock connection 15. The system has a disposable syringe 5 that is set on, a T-shaped connection piece 6 with an integrated pressure relief valve 7 controlled by a helical spring, to limit the pressure, a vein cannula 14 for attachment of the vein transplant, as well as a container holder structured as an encapsulated bottle holder 6, with an upper part 1 that can be screwed onto a lower part 2. A container 18, in which an endothelium protection solution is contained, is set into the bottle holder 6. When vein transplants are rinsed, the endothelium protection solution is passed through the T-shaped connection piece 6. When a defined pressure is exceeded, the pressure relief valve 7 opens to the side, and the solution can flow off into a collection container (not shown), through the hose 8. The hose 8 can be shut off by way of a hose clamp 10, or its through-flow can be limited. The precise limited pressure for the pressure relief valve can be tested in a pre-clinical study, and is then a prerequisite for the selection of a suitable pressure relief valve 7. A vein cannula 14 with an integrated back-flow barrier 15 follows the pressure relief valve 7.

In use, the vein transplant can be attached to the vein cannula 14 using a suture that is usual in operations, for example. The perfusion solution is contained in a commercially available infusion bottle 18 having a stopper, upside down, in the upper part of the two-part encapsulated bottle holder 16, into the lower part 2 of which a two-channel perforation mandrel 3 (hidden in FIG. 3a) with filters (air/fluid) is integrated. The two parts 1, 2 are connected with one another by means of a multi-channel so-called saw-tooth thread 17 that can be rotated only in the direction of the perforation mandrel. Depending on the purpose of use, other connections are also possible, for example bayonet closures, simple plug-in connections, or normal threads.

By means of rotating the upper part of the bottle holder 1, the perforation mandrel 3 is introduced into the bottle 18 just before use. The bottle 18 itself cannot be removed from the bottle holder 16 by the user, neither before nor after perforation by the mandrel 3, since the saw-tooth thread 17 does not permit the holder 16 to be opened. The bottle holder 16 can be attached to a usual infusion stand for operations, by way of a hanging device 19, or can be set onto a special stand (not shown).

The perfusion solution in the bottle holder 6 is connected with the membrane-controlled, automatic two-way valve 4 that is affixed between the syringe 5 and the pressure relief valve 7, by way of a hose connection 12. This two-way valve 4, which replaces a manually operated multi-way cock, allows the perfusion solution to be drawn into the syringe 5 and the vein segment to be subsequently rinsed, by emptying the syringe 5, without any additional manual intervention.

Use can proceed as follows: The vein transplant is first attached to the cannula 14 and subsequently rinsed, in order to fill it with rinsing solution and remove residues of blood. For a leak test of the vein segment, the distal end of the vein transplant is closed with a clamp that is usual for operations. Subsequently, it is rinsed again, under limited pressure. Non-tight side branches are recognized because rinsing solution exits from them, and can be surgically clamped off.

The vein segment, including the vein cannula 14, can be uncoupled from the remainder of the device and placed in a kidney bowl that is usual in operations, in epithelium protection solution, until reimplantation in the heart takes place. The back-flow barrier 11 integrated into the vein cannula 14 prevents back-flow of the solution out of the vein segment during this time, so that the blood vessel remains filled with the solution at slight excess pressure, and the endothelium layers that lie opposite one another do not come into contact, thereby protecting them.

Subsequently, the vein segment can be coupled to the perfusion device again, without any problems, for a further leak test.

1-46. (canceled)

47: Perfusion device for hollow structures, which comprises the following:
a perfusion fluid displacement device,
a cannula,
a valve device disposed between the perfusion fluid displacement device and the cannula,
wherein
the perfusion device comprises a perfusion fluid feed line connector,
the valve device is a multi-way valve device by way of which a hydraulic connection can be established between the perfusion fluid displacement device and the perfusion fluid feed line connector, as well as between the perfusion fluid displacement device and the cannula, and
wherein at least one excess pressure limiter is disposed between the perfusion fluid displacement, device and the cannula.

48: Perfusion device according to claim 47, wherein the excess pressure limiter comprises at least one pressure relief valve that opens a drain opening if a threshold pressure is exceeded.

49: Perfusion device according to claim 47, wherein the perfusion fluid displacement device is a perfusion fluid syringe, and
the multi-way valve device automatically releases the hydraulic connection between perfusion fluid syringe and perfusion fluid feed line connector, when the perfusion fluid syringe is filled, and automatically closes it when the perfusion fluid syringe is pressed.

50: Perfusion device according to claim 47, which has a perfusion fluid reservoir unit connected with the perfusion fluid feed line connector, by way of a perfusion fluid feed line.

51: Perfusion device according to claim 50, which has tempering means for tempering perfusion fluid contained in the perfusion fluid reservoir unit.

52: Perfusion device according to claim 50, wherein the perfusion fluid reservoir unit has a container holder into which a perfusion fluid container can be inserted.

53: Perfusion device according to claim 52, wherein the container holder has a locking mechanism by which a perfusion fluid container that has been inserted can be locked into the container holder.

54: Perfusion device according to claim 47, which has shut-off means for shutting off discharge through the drain opening.

55: Perfusion device according to claim 47, wherein the excess pressure limiter is designed for opening a drain opening at one of two different threshold pressures, respectively.

56: Perfusion device according to claim 55, wherein the lower of the two threshold pressures lies in a range of 20 to 200 Torr, and the higher of the two threshold pressures lies in a range of 100 to 700 Torr.

57: Perfusion device according to claim 47, wherein the device has a modular structure and can be taken apart without tools.

58: Perfusion device according to claim 57, wherein connecting means are provided, by way of which the cannula can be removed.

59: Perfusion device according to claim 47, which comprises a check valve to prevent back-flow through the cannula (14) into the perfusion device.

60: Perfusion device according to claim 47, wherein the cannula has a fastening device for fixing a hollow structure in place on the cannula.

61: Perfusion device according to claim 47, wherein the cannula has at least one conical section.

62: Perfusion device according to claim 47, which furthermore comprises a manometer device for measuring the pressure that prevails at the pressure relief valve.

63: Method for perfusion of an explanted hollow organ, which comprises the following:
providing a perfusion device comprising a perfusion fluid displacement device, a cannula, a multi-way valve device disposed between the perfusion fluid displacement device and the cannula, wherein the perfusion device comprises a perfusion fluid feed line connector, and wherein at least one excess pressure limiter is disposed between the perfusion fluid displacement device and the cannula,
connecting the fluid feed line connector to a fluid fill line,
attaching the hollow organ to the cannula,
rinsing the attached hollow organ by establishing a hydraulic connection between the perfusion fluid displacement device and the perfusion fluid feed line connector through the multi-way valve device, filling the perfusion fluid displacement device, establishing a hydraulic connection between the perfusion fluid displacement device and the cannula through the multi-way valve device.

64. Method according to claim 63, including a leak test of the hollow organ, wherein the distal end of the hollow organ is closed with a clamp.

65. Method according to claim 63, wherein the hollow organ, including the cannula, is filled with perfusion fluid and uncoupled from the remainder of the perfusion device after rinsing,
and wherein the cannula is provided with a back-flow barrier integrated therein to prevent back-flow of the perfusion solution out of the hollow organ.

66. Method according to claim 65, wherein the hollow organ is coupled to the perfusion device again for a further leak test.

67. Method according to claim 63, wherein an endothelium protection solution is used as the perfusion fluid.

68. Method according to claim 63, wherein a tempered perfusion fluid is used.
